(19) United States
(12) Patent Application Publication
(10) Pub. No.: US 2026/0066067 A1
(43) Pub. Date: Mar. 5, 2026

(54) SYSTEM AND METHOD FOR MATCHING A PATIENT MEDICAL CONDITION WITH A HEALTH CARE PROVIDER

(71) Applicant: BIOTRONIK SE & Co. KG, Berlin (DE)

(72) Inventors: Thomas DOERR, Berlin (DE); Volker LANG, Berlin (DE); Bjoern DIEM, Berlin (DE)

(73) Assignee: BIOTRONIK SE & Co. KG, Berlin (DE)

(21) Appl. No.: 19/104,033

(22) PCT Filed: Aug. 8, 2023
(86) PCT No.: PCT/EP2023/071949

§ 371 Date: Feb. 14, 2025

(30) Foreign Application Priority Data
Aug. 29, 2022 (EP) 22192522.5

(51) Int. Cl. G16H 10/60; G16H 40/20; G16H 50/20; G16H 80/00

(52) U.S. Cl. CPC G16H 10/60; G16H 40/20; G16H 50/20; G16H 80/00

(57) ABSTRACT

A data management system for matching a patient medical condition with a corresponding health care provider, including a computing device configured to apply an algorithm to the patient medical data to determine if the patient medical condition requires treatment, wherein if treatment of the patient medical condition is determined to be required, the algorithm is configured to match the patient medical condition with the corresponding health care provider according to the determined patient medical condition, and wherein the computing device is configured to identify and schedule a sequence of necessary medical treatments. A computer-implemented method for matching a patient medical condition with a corresponding health care provider is also provided.

### CROSS-REFERENCE TO RELATED APPLICATIONS

This application is the United States National Phase under 35 U.S. C. § 371 of PCT International Patent Application No. PCT/EP2023/071949, filed on Aug. 8, 2023, which claims the benefit of European Patent Application No. 22192522.5, filed on Aug. 29, 2022, the disclosures of which are hereby incorporated by reference herein in their entireties.

### TECHNICAL FIELD

The invention relates to a data management system for matching a patient medical condition with a corresponding health care provider.

### BACKGROUND

Furthermore, the invention relates to a computer-implemented method for matching a patient medical condition with a corresponding health care provider.

Current systems for referring patients are inefficient and lengthy. It often fails to select the appropriate treatment partner for the patient's current situation.

U.S. Pat. No. 9,504,423 B1 discloses a health monitoring apparatus comprising a comparison module configured to compare the physiology to the medical diagnostic criteria and to generate a diagnostic overread that comprises one or more diagnostic findings, and a referral module configured to determine the severity of any medical condition indicated by the diagnostic findings and to refer the patient to one or more pre-identified care providers via the computer for facilitated diagnosis as conditioned upon the severity of each such medical condition that exceeds a threshold tolerance level.

U.S. Pat. No. 2013/282391 A1 discloses a non-transitory computer-readable storage media having embodied thereon computer-executable instructions that, when executed by a server, perform a method of presenting patient-appropriate referral orders on a user interface associated with a patient portal, the method comprising receiving, from a first medical organization, a first referral order for a patient, the first referral order including referral information that is relevant to a clinician caring for the patient.

The method further comprises identifying a subset of the referral information of the first referral order that is relevant to the patient, the subset of the referral information of the first referral order comprising a first set of patient-appropriate information associated with the first referral order, wherein the first set of patient-appropriate information includes at least one of a referring provider name, a referral provider name, a reason for the referral, and an appointment date and time, extracting the first set of patient-appropriate information from the first referral order and generating a first modified referral order using the extracted first set of patient-appropriate information.

The present disclosure is directed toward overcoming one or more of the above-mentioned problems, though not necessarily limited to embodiments that do.

### SUMMARY

It is therefore an object of the present invention to provide an improved method for assessing a patient's medical condition and treating the same providing an enhanced diagnostic and treatment accuracy by assisting in a decision-making process of a health care provider.

The object is solved by a data management system for matching a patient medical condition with a corresponding health care provider having the features of claim 1.

Furthermore, the object is solved by a computer-implemented method for matching a patient medical condition with a corresponding health care provider having the features of claim 13.

Moreover, the object is solved by a computer program having the features of claim 14 and a computer-readable data carrier having the features of claim 15.

Further developments and advantageous embodiments are defined in the dependent claims.

The present invention provides a data management system for matching a patient medical condition with a corresponding health care provider, comprising at least one data storage device configured to store patient medical data comprising a plurality of medical parameters acquired by a patient sensor and health care provider data comprising information on health care providers qualified to treat medical conditions associated with said patient medical data.

Furthermore, the data management system comprises a first software interface to patient sensor, said first software interface being configured to receive the patient medical data and to transmit said patient medical data to the at least one data storage device and a second software interface to a further data storage device of the health care provider, wherein the second software interface is configured to update health care provider information in the at least one data storage device.

The data management system moreover comprises a computing device configured to apply an algorithm to the patient medical data to determine if the patient medical condition requires treatment, wherein if treatment of the patient medical condition is determined to be required, the algorithm is configured to match the patient medical condition with the corresponding health care provider according to the determined patient medical condition, and wherein the computing device is configured to identify and schedule a sequence of necessary medical treatments.

The present invention further provides a computer-implemented method for matching a patient medical condition with a corresponding health care provider. The method comprises storing patient medical data comprising a plurality of medical parameters acquired by a patient sensor and health care provider data comprising information on health care providers qualified to treat medical conditions associated with said patient medical data in at least one data storage device.

In addition, the method comprises receiving the patient medical data and transmitting said patient medical data to the least one data storage device by means of a first software interface to a patient sensor and updating health care provider information in the at least one data storage device by means of a second software interface to a further data storage device of the health care provider.

The method furthermore comprises applying an algorithm to the patient medical data to determine if the patient medical condition requires treatment by the health care provider, wherein if treatment of the patient medical condition is determined to be required, the algorithm matches the patient medical condition with the corresponding health care provider according to the determined patient medical condition, and wherein the computing device identifies and schedules a sequence of necessary medical treatments.

Furthermore, the present invention provides a computer program with program code to perform the method of the present invention when the computer program is executed on a computer. In addition, the present invention provides a computer-readable data carrier containing program code of a computer program for performing the method of the present invention when the computer program is executed on a computer.

The medical parameters are e.g. arrhythmia data, a heart rate, a patient activity, a chest impedance, and/or readings from electrodes of the implantable medical device.

An idea of the present invention is to ensure the assignment of patients to treatment providers according to need and without unnecessary delays and expenses.

In addition, due to the fact that the computing device is configured to identify and schedule a sequence of necessary medical treatments, i.e. the computing device assigning the patient to one or more healthcare providers in a specific sequence, the solution according to the invention advantageously enables patients to be assigned to treatment providers quickly, efficiently and in line with requirements.

According to an aspect of the invention, the patient sensor is a diagnostic implant and/or a therapeutic implantable medical device, in particular a loop recorder, an implantable pulse generator, an implantable cardioverter-defibrillator, a drug pump, a biochemical sensor and/or a cardiac resynchronization therapy device, and/or a wearable medical device, an environmental sensor and/or an external medical or therapy device.

Thus, data from a plurality of different sensor sources can be used to gather patient medical data, which in turn is used for matching a patient medical condition with a corresponding health care provider.

According to a further aspect of the invention, the at least one data storage device comprises a third software interface for depositing and/or updating patient electronic health record data and a fourth software interface configured to authenticate the patient electronic health record data and send it to a selected health care provider. The healthcare provider is thus able to remotely deposit and/or update patient electronic health record data.

According to a further aspect of the invention, the computing device is configured to determine whether on-site or remote treatment of the medical condition associated with said patient medical data is required and/or probable. This determination is performed by an algorithm operating on the computing device.

According to a further aspect of the invention, the at least one data storage device comprises a fifth software interface configured to receive a notification of the health care provider to accept, reject or delegate a treatment request, to revise the determination of the computing device, whether on-site or remote treatment of the medical condition associated with said patient medical data is required and/or to request further patient information, in particular patient symptoms.

The healthcare provider thus has a level of control over the determined health condition and recommended action determined by the algorithm in that the healthcare provider may agree, disagree or delegate or amend the treatment request.

According to a further aspect of the invention, the algorithm is configured to send the patient medical data comprising the plurality of medical parameters acquired by the patient sensor to a selected health care provider. The healthcare provider thus advantageously not only receives the determined health condition of the patient and the recommended action but also the raw data on which the decision of the algorithm is based. The healthcare provider is thus provided with sufficient supporting data to make a decision on a specific action to take.

According to a further aspect of the invention, the at least one data storage device comprises a sixth software interface configured to collect treatment metadata of the plurality of health care providers, in particular comprising a qualification, a ranking and or an availability for each of the plurality of health care providers. The algorithm can thus take said treatment metadata into account when making recommendations for healthcare providers to treat a specific health condition. Based on the treatment urgency of the determined health condition the availability of the healthcare provider is thus also taken into account by the algorithm when making a decision to recommend a specific healthcare provider.

According to a further aspect of the invention, the plurality of health care providers are physicians, therapists, clinics and/or emergency services and wherein the at least one data storage device is connected to an app on a patient communication device, in particular a smartphone and/or tablet computing device, communicating with the patient sensor. The patient communication device receiving medical data from the patient sensor is thus communicatively coupled with the data storage device of the data management system.

According to a further aspect of the invention, the at least one data storage device comprises a seventh software interface for depositing and/or updating payer information and/or preferences and an eighth software interface for depositing and/or updating patient treatment preferences. The data storage device thus advantageously further comprises interfaces that enable receiving additional information from the healthcare provider that cannot be determined from the patient sensor.

According to a further aspect of the invention, the computing device is configured to, based on the patient medical data comprising a plurality of medical parameters acquired by the patient sensor, determine a treatment urgency by the health care provider. This aspect thus contributes to enhancing patient safety in that should it be necessary an expedited treatment of the underlying health condition of the patient can be implemented.

According to a further aspect of the invention, the at least one data storage device comprises a ninth software interface and data for creating a quality index of the plurality of health care providers, in particular a treatment quality, a treatment cost, a reliability, a patient satisfaction and/or a payer ranking. That information can thus also be taken into account by the algorithm when making a recommendation for treating the patient condition.

According to a further aspect of the invention, the at least one data storage device is configured to store treatment price and/or billing models for healthcare providers, healthcare providers selection criteria and/or compliance rules for healthcare providers selection and comprises a tenth software interface for billing digital services.

Price and/or billing criteria can thus also be taken into account by the algorithm when making a recommendation for treating the given patient condition. Said economic criteria thus also contribute to the technical nature of the invention in that the most suitable healthcare provider is selected capable of treating the underlying health condition. Based on said further criteria, the algorithm thus has an additional data source assisting in determining the best possible healthcare provider for the determined health condition of the patient as treatment cost may correlate to treatment effectiveness.

The herein described features of the data management system for matching a patient medical condition with a corresponding health care provider are also disclosed for the computer-implemented method for matching a patient medical condition with a corresponding health care provider and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a diagram of a data management system for matching a patient medical condition with a corresponding health care provider according to a preferred embodiment of the invention; and

FIG. 2 shows a flowchart of a computer-implemented method for matching a patient medical condition with a corresponding health care provider according to the preferred embodiment of the invention.

### DETAILED DESCRIPTION

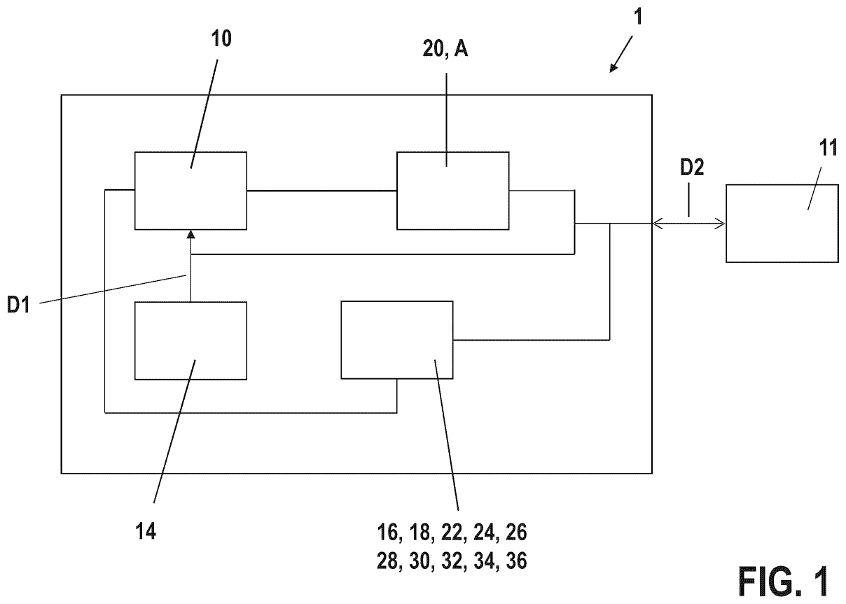

The data management system 1 shown in FIG. 1 for matching a patient medical condition with a corresponding health care provider comprises at least one data storage device 10 configured to store patient medical data D1 comprising a plurality of medical parameters 12 acquired by a patient sensor 14 and health care provider data D2 comprising information on health care providers qualified to treat medical conditions associated with said patient medical data D1.

Furthermore, the data management system 1 comprises a first software interface 16 to patient sensor 14, said first software interface 16 being configured to receive the patient medical data D1 and to transmit said patient medical data D1 to the at least one data storage device 10 and a second software interface 18 to a further data storage device 11 of the health care provider, wherein the second software interface 18 is configured to update health care provider information in the at least one data storage device 10.

The data management system 1 moreover comprises a computing device 20 configured to apply an algorithm A to the patient medical data D1 to determine if the patient medical condition requires treatment, wherein if treatment of the patient medical condition is determined to be required, the algorithm A is configured to match the patient medical condition with the corresponding health care provider according to the determined patient medical condition, and wherein the computing device 20 is configured to identify and schedule a sequence of necessary medical treatments.

If a relevant deviation in the patient medical data D1 is detected via the patient sensor 14 at the patient, this is evaluated by the computing device 20. The detection via a relevant deviation can take place in the patient sensor 14 at the patient and/or in the computing device 20.

The patient sensor 14 is a diagnostic implant and/or a therapeutic implantable medical device, in particular a loop recorder, an implantable pulse generator, an implantable cardioverter-defibrillator, a drug pump, a biochemical sensor and/or a cardiac resynchronization therapy device, and/or a wearable medical device, an environmental sensor and/or an external medical or therapy device.

The at least one data storage device 10 comprises a third interface for depositing and/or updating patient electronic health record data and a fourth interface configured to authenticate the patient electronic health record data and send it to a selected health care provider.

Moreover, the computing device 20 is configured to determine whether on-site or remote treatment of the medical condition associated with said patient medical data D1 is required and/or probable.

The at least one data storage device 10 further comprises a fifth interface configured to receive a notification of the health care provider to accept, reject or delegate a treatment request, to revise the determination of the computing device 20, whether on-site or remote treatment of the medical condition associated with said patient medical data D1 is required and/or to request further patient information, in particular patient symptoms.

The algorithm A is further configured to send the patient medical data D1 comprising the plurality of medical parameters 12 acquired by the patient sensor 14 to a selected health care provider. In addition, the at least one data storage device 10 comprises a sixth interface configured to collect treatment metadata of the plurality of health care providers, in particular comprising a qualification, a ranking and or an availability for each of the plurality of health care providers.

The plurality of health care providers are physicians, therapists, clinics and/or emergency services and wherein the at least one data storage device 10 is connected to an app on a patient communication device, in particular a smartphone and/or tablet, communicating with the patient sensor 14.

The at least one data storage device 10 comprises a seventh interface for depositing and/or updating payer information and/or preferences and an eighth interface for depositing and/or updating patient treatment preferences.

The computing device 20 is further configured to, based on the patient medical data D1 comprising a plurality of medical parameters 12 acquired by a patient sensor 14, determine a treatment urgency by the health care provider.

The at least one data storage device 10 comprises a ninth interface and data for creating a quality index of the plurality of health care providers, in particular a treatment quality, a treatment cost, a reliability, a patient satisfaction and/or a payer ranking.

The at least one data storage device 10 is configured to store treatment price and/or billing models for healthcare providers, to store healthcare providers selection criteria and/or compliance rules for healthcare providers selection and comprises a tenth software interface 36 for billing digital services. In addition, the at least one data storage device 10 further comprises a module for automatic verification of compliance rules in health care provider selection.

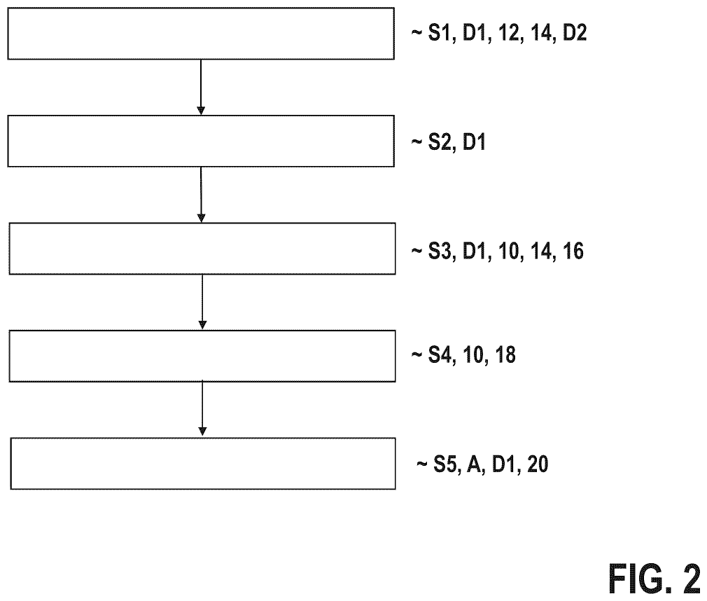

FIG. 2 shows a flowchart of a computer-implemented method for matching a patient medical condition with a corresponding health care provider according to the preferred embodiment of the invention.

The method comprises storing S1 patient medical data D1 comprising a plurality of medical parameters 12 acquired by a patient sensor 14 and health care provider data D2 comprising information on health care providers qualified to treat medical conditions associated with said patient medical data D1 in at least one data storage device 10.

Furthermore, the method comprises receiving S2 the patient medical data D1 and transmitting S3 said patient medical data D1 to the least one data storage device 10 by means of a first software interface 16 to a patient sensor 14 and updating S4 health care provider information in the at least one data storage device 10 by means of a second software interface 18 to a further data storage device 11 of the health care provider.

The method moreover comprises applying S5 an algorithm A to the patient medical data D1 to determine if the patient medical condition requires treatment by the health care provider, wherein if treatment of the patient medical condition is determined to be required, the algorithm A matches the patient medical condition with the corresponding health care provider according to the determined patient medical condition, and wherein the computing device 20 identifies and schedules a sequence of necessary medical treatments.

Although specific embodiments have been illustrated and described herein, it will be understood by those skilled in the art that a variety of alternative and/or equivalent implementations exist. It should be noted that the exemplary embodiment or exemplary embodiments are examples only and are not intended to limit the scope, applicability, or configuration in any way.

Rather, the foregoing summary and detailed description provides those skilled in the art with convenient guidance for implementing at least one exemplary embodiment, it being understood that various changes in the functional scope and arrangement of the elements may be made without departing from the scope of the appended claims and their legal equivalents.

In general, this application intends to cover modifications or adaptations or variations of the embodiments disclosed herein. For example, an order of the method steps may be modified. The method may further be carried out sequentially or in parallel, at least in part.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### Reference Signs

1 system

10 data storage device

11 further data storage device

12 plurality of medical parameters

14 patient sensor

16 first software interface

18 second software interface

20 computing device

22 third software interface

24 fourth software interface

26 fifth software interface

28 sixth software interface

30 seventh software interface

32 eighth software interface

34 ninth software interface

36 tenth software interface

A algorithm

D1 patient medical data

D2 health care provider data

S1-S5 method steps

1. Data management system for matching a patient medical condition with a corresponding health care provider, comprising:
at least one data storage device configured to store patient medical data comprising a plurality of medical parameters acquired by a patient sensor and health care provider data comprising information on health care providers qualified to treat medical conditions associated with said patient medical data;
a first software interface to patient sensor, said first software interface being configured to receive the patient medical data and to transmit said patient medical data to the at least one data storage device;
a second software interface to a further data storage device of the health care provider, wherein the second software interface is configured to update health care provider information in the at least one data storage device; and
a computing device configured to apply an algorithm to the patient medical data to determine if the patient medical condition requires treatment, wherein if treatment of the patient medical condition is determined to be required, the algorithm is configured to match the patient medical condition with the corresponding health care provider according to the determined patient medical condition, and wherein the computing device is configured to identify and schedule a sequence of necessary medical treatments.

2. Data management system of claim 1, wherein the patient sensor is a diagnostic implant and/or a therapeutic implantable medical device, in particular a loop recorder, an implantable pulse generator, an implantable cardioverter-defibrillator, a drug pump, a biochemical sensor and/or a cardiac resynchronization therapy device, and/or a wearable medical device, an environmental sensor and/or an external medical or therapy device.

3. Data management system of claim 1, wherein the at least one data storage device comprises a third software interface for depositing and/or updating patient electronic health record data and a fourth software interface configured to authenticate the patient electronic health record data and send it to a selected health care provider.

4. Data management system of claim 1, wherein the computing device is configured to determine whether on-site or remote treatment of the medical condition associated with said patient medical data is required and/or probable.

5. Data management system of claim 4, wherein the at least one data storage device comprises a fifth software interface configured to receive a notification of the health care provider to accept, reject or delegate a treatment request, to revise the determination of the computing device whether on-site or remote treatment of the medical condition associated with said patient medical data required and/or to request further patient information, in particular patient symptoms.

6. Data management system of claim 1, wherein the algorithm is configured to send the patient medical data comprising the plurality of medical parameters acquired by the patient sensor to a selected health care provider.

7. Data management system of claim 1, wherein the at least one data storage device comprises a sixth software interface configured to collect treatment metadata of the plurality of health care providers, in particular comprising a qualification, a ranking and or an availability for each of the plurality of health care providers.

8. Data management system of claim 1, wherein the plurality of health care providers are physicians, therapists, clinics and/or emergency services and wherein the at least one data storage device is connected to an app on a patient communication device, in particular a smartphone and/or tablet, communicating with the patient sensor.

9. Data management system of claim 1, wherein the at least one data storage device comprises a seventh software interface for depositing and/or updating payer information and/or preferences and an eighth software interface for depositing and/or updating patient treatment preferences.

10. Data management system of claim 1, wherein the computing device is configured to, based on the patient medical data comprising a plurality of medical parameters acquired by a patient sensor, determine a treatment urgency by the health care provider.

11. Data management system of claim 1, wherein the at least one data storage device comprises a ninth software interface and data for creating a quality index of the plurality of health care providers, in particular a treatment quality, a treatment cost, a reliability, a patient satisfaction and/or a payer ranking.

12. Data management system of claim 1, wherein the at least one data storage device is configured to store treatment price and/or billing models for healthcare providers, to store healthcare providers selection criteria and/or compliance rules for healthcare providers selection and comprises a tenth software interface for billing digital services.

13. Computer-implemented method for matching a patient medical condition with a corresponding health care provider, comprising the steps of:
storing patient medical data comprising a plurality of medical parameters acquired by a patient sensor and health care provider data comprising information on health care providers qualified to treat medical conditions associated with said patient medical data in at least one data storage device;
receiving the patient medical data and transmitting said patient medical data to the least one data storage device by means of a first software interface to a patient sensor;
updating (S4) health care provider information in the at least one data storage device (10) by means of a second software interface (18) to a further data storage device (11) of the health care provider; and
applying (S5) an algorithm (A) to the patient medical data (D1) to determine if the patient medical condition requires treatment by the health care provider, wherein if treatment of the patient medical condition is determined to be required, the algorithm (A) matches the patient medical condition with the corresponding health care provider according to the determined patient medical condition, and wherein the computing device (20) identifies and schedules a sequence of necessary medical treatments.

14. Computer program with program code to perform the method of claim 13 when the computer program is executed on a computer.

15. Computer-readable data carrier containing program code of a computer program for performing the method of claim 13 when the computer program is executed on a computer.
